(19) United States
(12) Patent Application Publication
(10) Pub. No.: US 2025/0177725 A1
(43) Pub. Date: Jun. 5, 2025

(54) BLOOD PUMP WITH CANTILEVERED IMPELLER

(71) Applicant: Shifamed Holdings, LLC, Campbell, CA (US)

(72) Inventors: Michael CALOMENI, San Jose, CA (US); Ari RYAN, San Jose, CA (US); Crissly CRISOSTOMO, Campbell, CA (US); Gregory Michael HAMEL, Redwood City, CA (US); Daniel HILDEBRAND, Santa Cruz, CA (US); Seyed Mostafa GHOREYSHI, Campbell, CA (US); Janine ROBINSON, Half Moon Bay, CA (US)

(21) Appl. No.: 18/841,344

(22) PCT Filed: Feb. 27, 2023
(86) PCT No.: PCT/US2023/063381

§ 371 Date: Aug. 23, 2024

Related U.S. Application Data
(60) Provisional application No. 63/268,503, filed on Feb. 25, 2022.

(51) Int. Cl. A61M 60/804; A61M 60/13; A61M 60/174; A61M 60/216; A61M 60/414; A61M 60/81; A61M 60/825

(52) U.S. Cl. CPC A61M 60/804; A61M 60/13; A61M 60/174; A61M 60/216; A61M 60/414; A61M 60/81; A61M 60/825

(57) ABSTRACT

Intravascular blood pumps systems and methods of use are provided. The blood pump system includes a catheter portion having a distal blood pump with a cantilevered collapsible impeller. The blood pump includes sections of increased stiffness and sections of increased flexibility. The blood pump is designed and configured to bend at a distal tip, a central section, and in a proximal catheter shaft, while maintaining stiffness in the inlet and outlet. Collectively the pump is designed and configured to maintain tip gap between the impeller and the blood conduit without requiring any structures in the blood conduit distal to the impeller assembly.

### CROSS REFERENCE TO RELATED APPLICATIONS

This application claims the benefit of priority to U.S. Application No. 63/268,503, filed Feb. 25, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in combination with pharmacological treatment.

Catheter blood pumps have been known for support of hemodynamically unstable patients for decades. Catheter blood pumps are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

One such blood pump, the Hemopump, was developed in 1980's as the first percutaneous blood pump. The Hemopump included a rotary pump guided by a catheter with a long, flexible inflow extension. The Hemopump had an axial flow design to improve flow efficiency for the given catheter profile. The Hemopump was expanded to several sizes. However, the smaller size of 14Fr required a surgical cutdown and provided inadequate flow. The 21Fr version had increased risk of complications and limited additional flow.

Efforts have been made to provide higher flow while limiting the introduction profile of the catheter. Examples include the expandable pump shown in U.S. Pat. No. 5,749,855 to Reitan (“the '855 patent”). The '855 patent describes an expandable impeller within protective outer filaments. The impeller blades are hinged to allow the entire pump to be collapsed for introduction and expanded within the body. However, the hydraulic efficiency suffers because the impeller rotates within the large vessel and thus cannot generate pressure to create flow.

Other examples include U.S. Pat. No. 7,393, 181 to McBride (“the '181 patent”), U.S. Pat. No. 9,446,179 to Keenan (“the '179 patent”), U.S. Pat. No. 9,512,839 to Liebing (“the '839 patent”), and U.S. Pat. No. 6,533,716 to Schmitz-Rode (“the '716 patent”). These references all describe expandable pumps formed with expandable impellers within expandable shrouds. However, these pumps involved complicated designs.

For example: the '716 patent provides a design with an unsupported flow lumen. With no support in the flow lumen, the blood pump lacks hydraulic efficiency and risks impeller rubbing on or contacting an inner diameter of the flow lumen. The '181 patent and similar pumps require a drive shaft to extend through the impeller and terminate at a bearing at a distal end of the shroud. The '179 patent and the '839 patent both describe a distal bearing within the shroud. Each of these require extra moving parts, which increases hemolysis and the risk of thromboembolic events. The additional structure(s) also add or lengthen sections of increased stiffness in the pump, making it harder to introduce the pump and causing the pump to be stiff across the aortic valve and within the ventricle. The '839 patent also involves placing the high-speed impeller in the ventricle which is believed to cause risk of complications.

There is a need for a true percutaneous pump that can provide adequate cardiac support for a variety of heart failure populations. There is a need for a pump that is easy to introduce and track, and that is adaptable to the anatomy. There is a need for a pump with reduced risk of injury and complications.

### SUMMARY OF THE DISCLOSURE

The disclosure is related to intravascular blood pump and methods of their use.

An expandable catheter blood pump is provided, comprising a blood conduit supported by a catheter, the blood conduit comprising an expandable scaffold and an impermeable membrane disposed on a portion of the expandable scaffold; an impeller assembly rotatably supported within a proximal portion of the blood conduit; wherein the impeller assembly is the only structure positioned within the blood conduit.

In some embodiments, the impeller assembly includes one or more impeller blades coupled to an impeller shaft.

In one aspect, the impeller shaft passes through an impeller bearing assembly proximal to the one or more impeller blades.

In some aspects, the impeller bearing assembly is at least partially positioned within a proximal hub of the blood pump.

In one embodiment, the blood pump further includes a flexible drive cable coupled to a proximal end of the impeller shaft.

In some aspects, the impeller shaft is stiffer than the flexible drive cable.

In one aspect, the impeller assembly is cantilevered.

In some embodiments, there are no structures positioned distal to the impeller assembly within the blood conduit.

In one aspect, the blood conduit comprises an open lumen distal to the impeller assembly.

In some embodiments, no bearings are positioned distal of the impeller assembly.

In one aspect, the impeller assembly is disposed within an outlet section of the expandable scaffold.

In some embodiments, the outlet section includes a plurality of proximal struts that form at least one opening.

In one embodiment, the at least one opening is positioned proximal to a proximal end of the impermeable membrane.

In some aspects, the impeller assembly is at least partially disposed within the outlet section.

In some embodiments, the expandable section further comprises a central portion distal to the impeller assembly.

In one embodiment, the central portion is more flexible than the outlet section.

In some embodiments, the catheter further comprises a distal shaft section coupled to the outlet section.

In one aspect, the distal shaft section is more flexible than the outlet section.

An expandable catheter blood pump is provided, comprising: a blood conduit supported by a catheter, the blood conduit comprising an expandable scaffold and an impermeable membrane disposed on the expandable scaffold, wherein the expandable scaffold includes five distinct sections including: a) a plurality of distal struts forming an inlet section, b) a distal shroud section, c) a central shroud section, d) a proximal shroud section, and e) a plurality of proximal struts forming an outlet section; an impeller disposed in the proximal shroud section and rotatably supported by a drive cable assembly that extends through the catheter, through the outlet section, and into the proximal shroud section, wherein the drive cable assembly is more flexible proximal to the outlet section than it is through the outlet section and proximal shroud section.

In some aspects, the drive cable assembly does not extend distally into the central shroud section.

In some embodiments, the impeller and the drive cable assembly are not positioned in the central shroud section, the distal shroud section, or the inlet section.

In one embodiment, the drive cable assembly comprises a flexible drive cable coupled to a rigid impeller shaft.

In some aspects, the flexible drive cable is coupled to the rigid impeller shaft proximal to an impeller bearing assembly.

In one embodiment, the flexible drive cable is more flexible than the rigid impeller shaft.

In some aspects, the drive cable assembly and impeller are cantilevered.

In some embodiments, there are no structures positioned within the blood conduit except for the impeller and drive cable assembly.

An expandable catheter blood pump is provided, comprising: a catheter shaft; a proximal hub assembly coupled to the catheter shaft; a blood conduit supported by the proximal hub, the blood conduit comprising an expandable scaffold and an impermeable membrane disposed on the expandable scaffold, the expandable scaffold comprising an inlet section, a central section, and an outlet section; a cantilevered impeller assembly disposed at least partially within the outlet section, the cantilevered impeller assembly including a drive assembly that extends through the catheter shaft and the impeller assembly; and a distal tip coupled to the inlet section; wherein the distal tip, central section, and catheter shaft are more flexible than the inlet section and the outlet section, wherein the blood pump is configured to bend in the distal tip, the central section, and/or the catheter shaft in response to forces applied to the blood pump.

In some embodiments, the outlet section and drive assembly are sufficiently stiff to maintain tip gap between the impeller assembly and the blood conduit when forces are applied to the blood pump.

In some aspects, the drive assembly is more flexible in the catheter shaft than it is in the outlet section.

In one embodiment, the drive assembly comprises a flexible drive cable in the catheter shaft and an impeller shaft in the outlet section.

In some aspects, the impeller shaft passes through an impeller bearing assembly.

In some embodiments, the drive assembly does not extend distally into the central section.

In one embodiment, the impeller assembly and the drive assembly are not positioned in the central section or the inlet section.

A catheter blood pump is provided, comprising: a blood conduit supported by a catheter, the blood conduit comprising an expandable scaffold and an impermeable membrane, the blood conduit having a substantially constant diameter; and an impeller rotatably supported within a proximal portion of the blood conduit, the impeller having a diameter that decreases from a proximal portion of the impeller to a distal portion of the impeller, the blood pump being configured to rotate the impeller within the blood conduit to pump blood from a blood pump input to a blood pump outlet.

### BRIEF DESCRIPTION OF THE DRAWINGS

FIGS. 1A-1B are side views of an exemplary blood pump that includes an expandable scaffold that supports a blood conduit with an impeller housed therein.

FIG. 2 shows a catheter shaft portion of a blood pump.

FIG. 3 shows a scaffold and blood conduit of a blood pump.

FIG. 4 is a cutaway view of an outlet section of a blood pump.

FIG. 5 shows a distal hub of a blood pump.

FIG. 6 shows a distal tip of a blood pump.

FIG. 7 shows a cutaway view of an impeller within an outlet section of a blood pump.

FIGS. 8A-8B show deflection of a blood pump in response to forces applied against the blood pump.

FIG. 9 shows one embodiment of a tapered impeller of a blood pump.

### DETAILED DESCRIPTION

Minimally-invasive rotary blood pumps are provided that can be inserted into the body in connection with the cardiovascular system to, e.g., pump arterial blood from the left ventricle into the aorta to add to the native blood pumping ability of the left side of the patient's heart. An overall goal for the use of such blood pumps is to reduce the workload on the patient's heart muscle to stabilize the patient, such as during a medical procedure that may put additional stress on the heart, to stabilize the patient prior to heart transplant, or for continuing support of the patient.

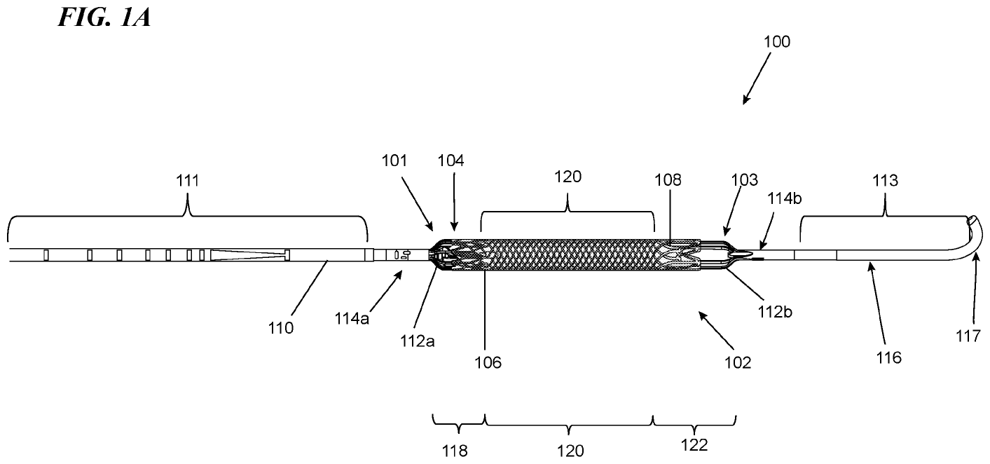

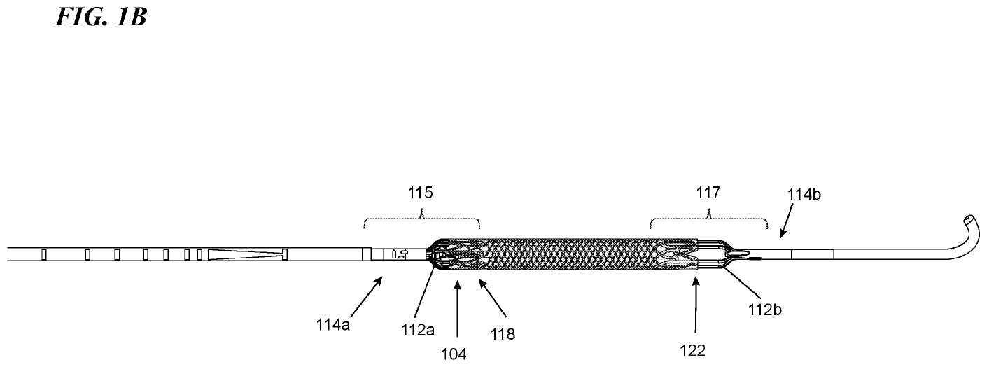

FIG. 1A shows an example of an intravascular blood pump 100. The blood pump 100 includes an expandable/collapsible blood conduit 102 that is configured to transition between an expanded state, as shown in FIG. 1A, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. When in the expanded state, the inner lumen of the conduit 102 may be configured to accommodate blood pumped by an impeller disposed therein.

In this example, the blood pump 100 includes an impeller 104 within a proximal portion of the conduit 102. The conduit 102 includes a first (e.g., proximal) end having a first (e.g., proximal) opening 101, and a second (e.g., distal) end having a second (e.g., distal) opening 103. The first opening 101 may be configured as an outlet and the second opening 103 may be configured as an inlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The impeller 104 may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The exemplary conduit 102 includes a tubular expandable/collapsible scaffold 106 that provides structural support for a membrane 108 that covers at least a portion of inner surfaces and/or outer surfaces of the scaffold 106. The scaffold 106 defines a supported lumen or blood conduit with radial strength to maintain blood flow during operation of the blood pump. The exemplary conduit is formed to be fluid impermeable by the membrane. The membrane may be attached to the scaffold, cover the scaffold, be sandwiched or molded around the scaffold, or integrated into the scaffold, and other configurations as would be understood by one of skill from the description herein. The exemplary scaffold 106 includes a material having a pattern or plurality of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nickel titanium (nitinol), cobalt alloys, and polymers, although other materials may be used.

The exemplary scaffold 106 includes proximal struts 112a extending at a proximal end near the first opening 101 (e.g., blood outlet region) and distal struts 112b that extend from the scaffold 106 near the second opening 103 (e.g., blood inlet region). The proximal struts 112a are coupled to first hub 114a of a shaft 110 of a catheter. The distal struts 112b are coupled to second hub 114b. The second or distal hub 114b can be coupled, connected to, or integral with an atraumatic distal tip 116. The distal tip 116 can be extremely flexible and compliant. In some examples, the distal tip can include a distal bend or curvature, just as a J-tip, a pigtail tip, or the like. In this example, the first hub 114a includes a bearing assembly through which a central drive cable extends. The drive cable is operationally coupled to and configured to rotate the impeller 104.

The conduit 102 has a proximal region 118, a central region 120, and a distal region 122. The central region 120 may be configured to be placed across a valve (e.g., aortic valve) such that the proximal region 118 is configured to be placed at least partially within a first heart region (e.g., ascending aorta) and the distal region 122 is at least partially within a second heart region (e.g., left ventricle). The proximal region 118 may be configured to house an impeller therein. As shown, the blood conduit 102 can have a substantially constant diameter.

The distal tip 116, conduit 102, and shaft 110 can each include sections of increased flexibility (e.g., less stiffness) relative to other sections of the blood pump. The increased flexibility sections are configured to deflect or bend when a load is applied to the blood pump 100. In some implementations, distal shaft portion 111 of shaft 110, central region 120 of conduit 102, and distal tip section 113 of distal tip 116 are the most flexible regions or sections of the blood pump 100.

For example, still referring to FIG. 1A, the shaft 110 can include a distal shaft portion 111 that is more flexible than other sections of the shaft 110. The distal shaft portion 111 can achieve increased flexibility by having a reduced braid density, reduced durometer, and/or reduced wall thickness compared to other sections of the shaft 110.

Additionally, the conduit 102/scaffold 106 can include a central region 120 that is more flexible than the proximal region 118 and distal region 122 of the conduit. The scaffold pattern/design has been optimized to give the central region 120 more flexibility and reduced stiffness relative to the rest of the scaffold. For example, the central region 120 can include a section of helically winding scaffold elements with an optimized pitch and decreased element width to facilitate flexibility and bending in response to the anatomy while resisting kinking or collapse of the conduit.

Furthermore, the distal tip 116 can include a distal tip section 113 that is also extremely flexible/compliant. The increased flexibility of the distal tip section 113 can be achieved with decreased durometer sections and/or decreased wall thickness. The length of the distal tip 116 is configured to allow the blood pump to “dock” or rest in the ventricle apex to reduce movement of the overall assembly during use. The flexible distal tip section 113 allows for bending and accommodation of heart contraction, pump movement and varying anatomy. Additionally, the distal tip section 113 can include varying wall thickness and durometer to optimize bending and to distribute loads to other sections of the blood pump 100 (e.g., to the central region 120 or the distal shaft portion 111.

While FIG. 1A and the discussion above describes sections of the blood pump 100 with increased flexibility or reduced stiffness relative to other sections of the blood pump, FIG. 1B shows outlet section 115 and inlet section 117 of the blow pump 100 with increased stiffness or reduced flexibility relative to other sections of the blood pump. Outlet section 115 can generally include proximal portion 118 of the conduit, impeller 104, proximal struts 112a, and hub 114a. Inlet section 117 can generally include distal portion 122 of the conduit, distal struts 112b, and hub 114b. Generally, outlet section 115 and inlet section 117 are stiffer than distal shaft portion 111 of shaft 110, central region 120 of conduit 102, and distal tip section 113 of distal tip 116.

FIGS. 1A and 1B show the sections of the blood pump with increased flexibility and the sections of the pump with increased stiffness. Collectively, these sections of stiffness and flexibility are designed and arranged to provide for stiffness in sections that promote concentricity of the impeller within the scaffold while allowing for flexibility in sections that contact the tissue or must bend around the anatomy. Generally, the inlet and outlet sections of the blood pump are relatively stiff compared to the central section of the scaffold, the distal tip, and the catheter shaft which are relatively flexible. This allows for a load applied to the distal tip (e.g., contacting the heart wall with the distal tip) to be transferred from the distal tip to the center of the scaffold (central region 120) to prevent bending at the hub or inlet section of the pump. Thus, the central region is designed and configured to always give in first when a side load is applied to the blood pump or scaffold (e.g., contacting the anatomy). In the inlet and outlet sections, the struts can have increased width near attachment to the hubs (e.g., hubs 114a and 114b) to stiffen the scaffold in those sections. The outlet section includes stiff radial and axial elements and rigid support in the impeller bearing assembly for the cantilevered impeller and impeller shaft design to promote concentricity of impeller and scaffold and maintain appropriate tip gap. Similarly, the inlet section can include an increase in width for distal elements to maintain the inlet and prevent collapse. Rigidity in the distal inlet section allows any potential force from distal tip to be translated to central portion of the scaffold (and not to the inlet section). The central section accommodates compound bending and pump movement while minimizing deflection to the outlet section on the proximal portion of the scaffold.

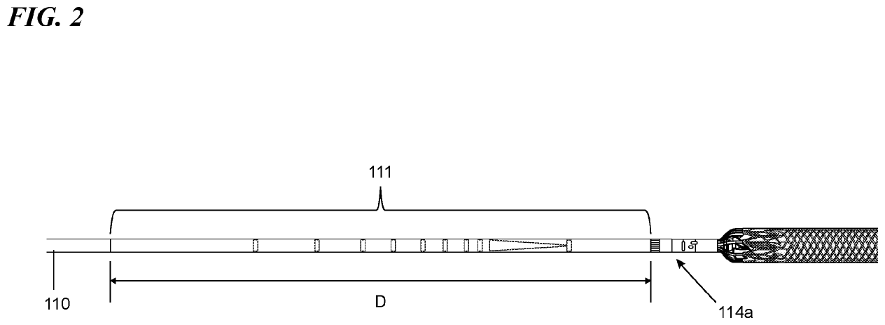

FIG. 2 shows a close-up view of distal shaft portion 111 of catheter 110, including the connection to hub 114a. FIG. 2 includes an illustration of the features that enable distal shaft portion 111 to be flexible relative to other sections of the shaft. Shaft 110 of the catheter is coupled to hub 114a of the blood pump, and extends proximally from the hub 114a to a handle and/or console that controls operation of the system. When the blood pump is inserted inside the heart of a patient, the shaft 110 extends proximally away from the pump and out of the body of the patient. In some embodiments, distal shaft portion 111 of the catheter is the most flexible section of the catheter 110. This increased flexibility relative to the rest of the catheter can be achieved, for example, with a reduced braid density, a reduced durometer, and/or a reduced wall thickness. This flexible distal shaft portion 111 is designed and configured such that its features align anatomically with the aortic arch and descending aorta when the blood pump is inserted into the heart of the patient. In some embodiments, the distal shaft portion 111 can have a length D. This length D can be, for example, up to 5″ in length. The distal shaft portion 111 can have an increased flexibility or reduced stiffness in this length D relative to the rest of the shaft 110. This results in reduced pressure against the leaflets and ventricular wall. The reduced external forces on the pump (pressure on ventricular wall and valve) aide in maintaining concentricity between the pump and scaffold. Additionally, the reduced pressure on the ventricular wall helps to maintain tip gap (i.e., the distance between the impeller and the scaffold/blood conduit) and reduces anatomical damage to the heart and surrounding anatomy.

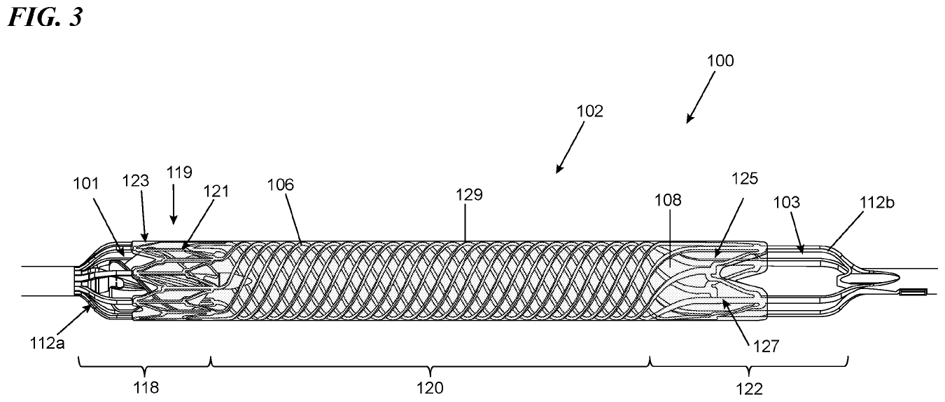

FIG. 3 shows an additional view of the blood conduit 102 of the blood pump 100, including scaffold 106, membrane 108, proximal struts 112a, first opening (outlet) 101, distal struts 112b, and second opening (inlet) 103. The blood conduit and/or scaffold of the blood pump can include features or design elements configured to maintain tip gap between the impeller and scaffold/membrane during use. As previously described, the conduit 102 can include a proximal region 118, a central region 120, and a distal region 122. The blood conduit 102 of the pump is designed with specific features configured to maintain tip gap between the impeller and the scaffold/membrane in varying anatomy. This includes design elements that allow specified sections or portions of the blood conduit to be flexible while other specified portions or sections of the blood conduit are stiff.

For example, the proximal portion 118 of the scaffold can include a section 119 of increased element density to increase stiffness around the impeller and the outlet section. The section 119 of increased element density can include a plurality of axial elements 121 connected to a plurality of radial elements 123. In some embodiments, the radial elements can be arranged in a chevron pattern or arrangement of diagonal elements. In the illustrated embodiment, the axial elements 121 are positioned between two sections of radial elements 123. In some embodiments, the radial and axial elements within the section 119 can have increased width relative to other elements in the scaffold (e.g., the helical elements in central section 120) to provide increased stiffness in section 119. Additionally, as shown in FIG. 3, the proximal struts 112a can have a tapered leg design with a wider base that is optimized for stiffness in the outlet region of the blood pump.

The distal portion 122 of the scaffold can also include a section 125 of increased element density to increase stiffness around the inlet section. As with the proximal portion, the distal portion can include a plurality of axial elements 127. The section 125 can include elements with an increased width relative to other elements in the scaffold (e.g., the helical elements in central section 120) to provide increased stiffness in section 125. Additionally, the distal struts 112b can have a tapered leg design with a wider base that is optimized for stiffness in the inlet region of the blood pump.

As also shown in FIG. 3, the central portion 120 can include a plurality of helical elements 129 with a pitch and width optimized to facilitate bending in the central portion without kinking. The central portion 120 must be sufficiently flexible so that it is softer than the anatomical structures it will be positioned against. Allowing for bending without kinking allows for flow performance to be maintained in a variety of operating conditions. Therefore, accommodating the anatomy does not come at the cost of flow performance. In some embodiments, the helical elements 129 have a width that is less than a width of elements in section 119 of the proximal portion 118 and section 125 of the distal portion 122.

In some aspects, the scaffold 106 shown in FIG. 3 can generally be divided into five distinct sections: 1) central portion 122, 2) section 125, 3) section 119, 4) distal strut section 112b, 5) proximal strut section 112a. As described above, the scaffold can include central portion 122 which can include flexible helical elements that are designed to bend without kinking or collapsing, allowing flow performance to be maintained in a variety of operating conditions. Accommodating the anatomy does not come at the cost of flow performance. The central section must be sufficiently flexible so that it's softer than anatomical structures and gives way when the blood pump contacts the anatomy. The scaffold further includes stiffer sections 119 (around the impeller 104) and 125 (towards the inlet section). Furthermore, the sections around the proximal and distal struts (the inflow and outflow sections) are also relatively stiff. In combination with sections 119 and 125, the sections on the proximal and distal ends of the scaffold function as a relatively stiff beam, especially compared to the flexible central portion. Thus, the central portion will always give or bend first when a side load is applied to the scaffold (e.g., contact with the anatomy).

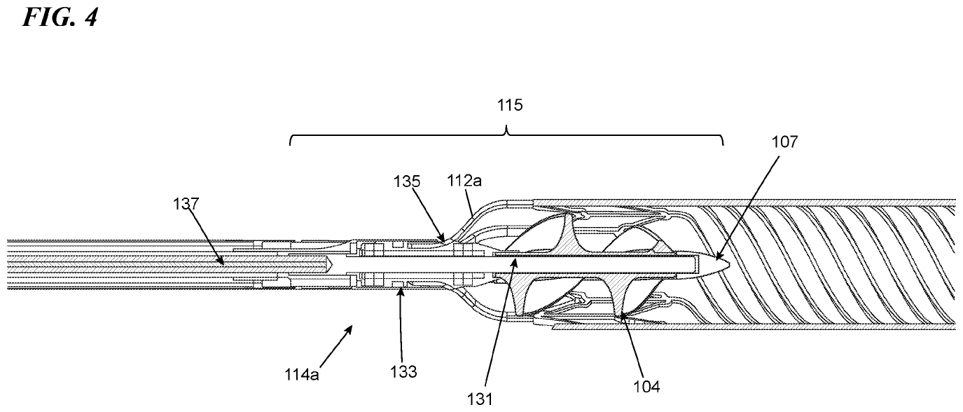

FIG. 4 is a closeup view of outlet section 115 of the blood pump, including features that provide increased stiffness. In this figure, impeller 104 is shown supported by impeller shaft 131, which extends through impeller bearing assembly 133 that is positioned within hub 114a and terminates at a distal end of impeller (optionally just distal to the impeller blades). Generally, the structures illustrated in FIG. 4 within the proximal portion of the blood conduit can collectively be referred to as the impeller assembly. The impeller assembly can include, for example, the impeller shaft, the impeller, any distal cap or hub on the distal end of the impeller shaft, and any structures that couple or encapsulate the proximal portion of the impeller shaft to the impeller bearing assembly. In some embodiments, a distal impeller hub 107 can seal, encapsulate, or cover the impeller shaft and/or impeller blades. While the distal impeller hub is shown as a distinct element in FIG. 4, it should be understood that in some embodiments there is not a distal impeller hub or optionally the distal impeller hub is integral to the impeller shaft. The distal end of the impeller is not supported by any other features or elements and is not connected to the scaffold or to a distal end of the blood pump or conduit. There are no structures positioned within the scaffold 106 distal to the impeller assembly (e.g., the impeller, impeller shaft, and optionally the distal impeller hub). In fact, in the embodiment of FIG. 4, the impeller assembly is the only structure positioned within the blood conduit of the blood pump. The impeller 104 and impeller shaft 131 are arranged in a cantilevered design configuration in which the distal end of the impeller assembly is freely positioned within the scaffold. The position of the impeller relative to the scaffold is maintained by structures in the outlet section of the blood pump and/or the configuration of the impeller and scaffold themselves. No distal bearing is required or used. The stiffness of the impeller shaft 131 and passage of the shaft through the stiff impeller bearing assembly 133 provides the stiffness required to maintain impeller position and tip gap within the scaffold and blood conduit during operation in a variety of conditions. The overall design including stiffness around the impeller provides a “quiet” section around the impeller to allow for maintaining tip gap while providing flexibility on either side of the impeller (e.g., in the catheter shaft and in the central portion of the scaffold). Preventing or limiting bending around the impeller and impeller shaft means that concentricity of the impeller and ultimately tip gap between the impeller and the scaffold is maintained. It is further noted that in some embodiments, the proximal struts 112a can be inserted into and supported by the impeller bearing assembly 133 in section 135. The supported struts 112a adds rigidity to the outlet section 115.

Drive cable 137 extends through the shaft and is coupled to the impeller shaft 131. The drive cable can be rotated by a motor (not shown) to provide rotation to the impeller. The drive cable can be sufficiently flexible to allow for the flexibility in distal shaft portion 111 described above. The attachment of the drive cable to the impeller shaft allows for increased stiffness through the hub 114a, impeller bearing assembly 133, and impeller 104.

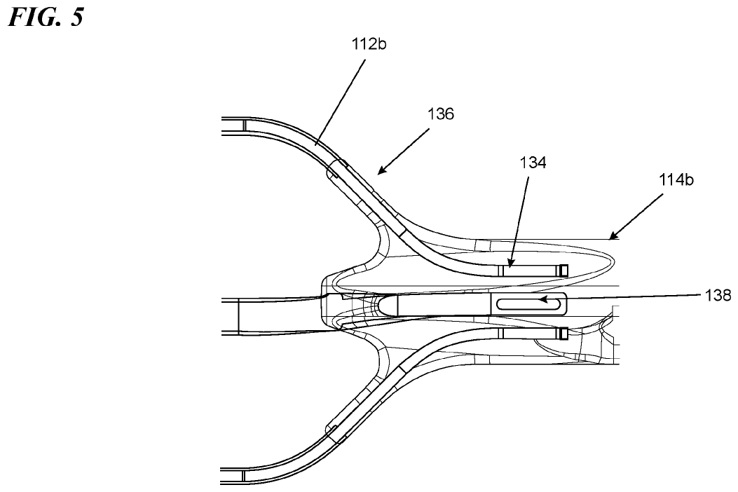

FIG. 5 illustrates a closeup view of distal hub 114b. In this embodiment, the struts can be overmolded into the hub itself. In this view, it can be seen how the distal ends 134 of struts 112b extend past arms 136 of the hub and into the hub body itself. Extension of the struts into the hub body increases stiffness of the hub (and therefore the inlet section) and can prevent buckling or bending at the strut/arm transition when large forces are applied to the blood pump (e.g., to the distal tip). The distal ends 134 of the struts can further include slits or through features 138.

The slits or through features 138 allow for flow-through of a polymer material(s) during injection molding of the hub to ensure a strong bond between the struts and the hub and to prevent the struts from withdrawing from the hub.

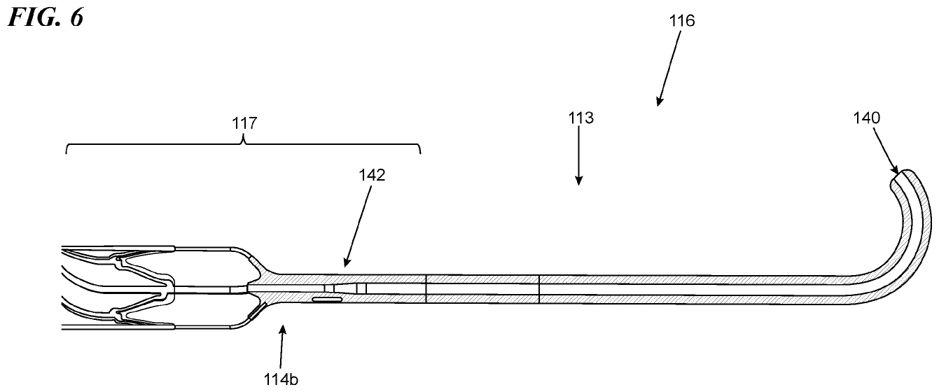

FIG. 6 shows a cross-sectional view of inlet section 117 and its connection to the distal tip 116, including flexible distal tip section 113. As described above, the flexible distal tip section 113 can be configured and designed to have increased flexibility by adjusting or changing the wall thickness within the tip. The distal tip 116 can include a guidewire lumen 140 that extends from the inlet section through the distal tip. The guidewire lumen can be configured to accommodate and receive a guidewire during positioning of the blood pump in the anatomy. As shown in FIG. 6, the guidewire lumen can include a junction 142 either within the hub 114b or at the transition from the hub to the distal tip section 113 in at which the diameter of the guidewire lumen changes. As shown, the diameter of the guidewire lumen within the hub 114b is smaller than the diameter of the guidewire lumen within the distal tip section 113. In the illustrated embodiment, the diameter of the guidewire lumen transitions from the first (smaller) diameter in the hub to the second (larger) diameter in the distal tip section. This results in a larger wall thickness in the hub, resulting in more stiffness in the hub, compared to a smaller wall thickness in the distal tip, resulting in less stiffness in the distal tip. In addition to using wall thickness to make the distal tip more flexible, the distal tip 116 can also comprise a material of reduced durometer to increase flexibility in the distal tip section 113.

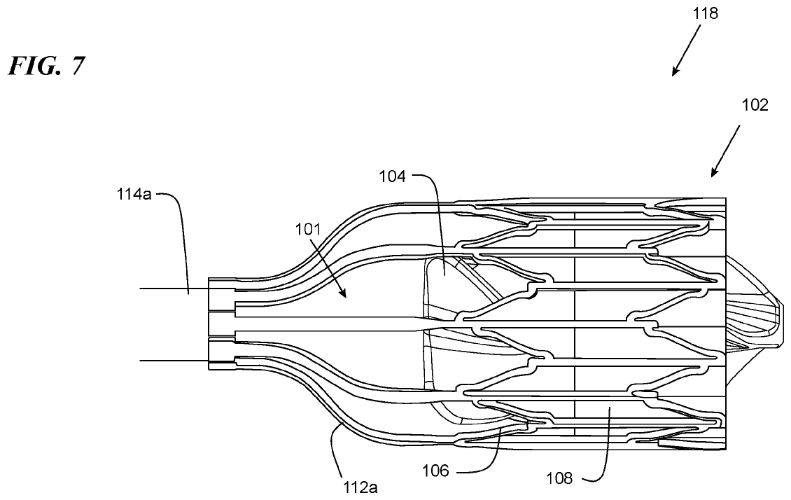

FIG. 7 shows a closeup view of the proximal portion 118 of the conduit 102, including impeller 104, scaffold 106, membrane 108, proximal struts 112a, and proximal hub 114a. In some cases, the impeller 104 is fully positioned axially within the conduit 102. In other cases, as shown in FIG. 2, a proximal portion of the impeller 104 is positioned at least partially outside of the conduit 102. That is, at least a portion of the impeller may extend proximally beyond a proximal end of the conduit. As shown in FIG. 7, a portion of the impeller 104 can be seen extending beyond the membrane/conduit and into the first opening 101 (e.g., extending into the outlet). When the impeller extends proximally past a proximal end of the conduit, the moment arm formed by the impeller and impeller shaft is reduced.

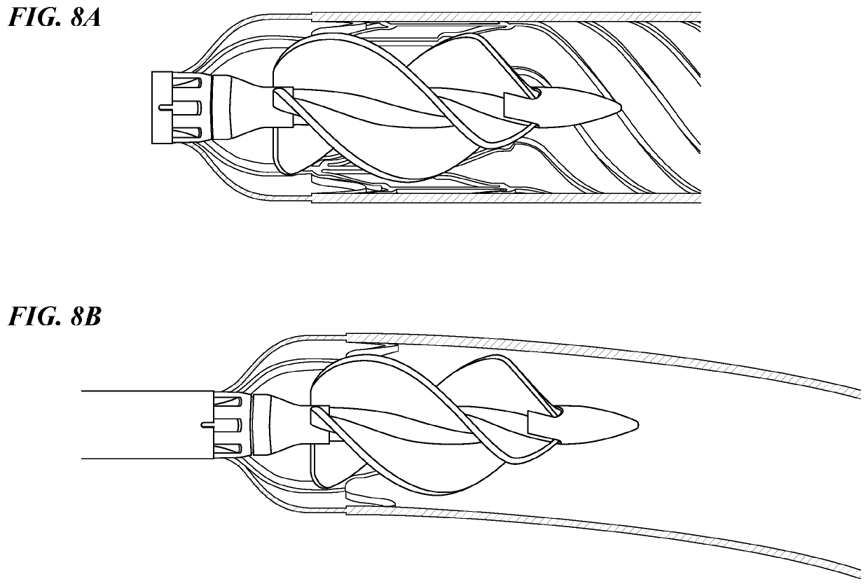

It is important to maintain a minimum distance between the rotating impeller 104 and the inside surface of the blood conduit 102 so that the impeller blades do not contact the inside surface of the blood conduit while they are rotating. When the blood conduit extends in a straight line, as shown in FIG. 8A, the impeller's longitudinal axis is centered on the longitudinal axis of the blood conduit, and the gap between the rotating impeller blades and the inside surface of the blood conduit remains substantially constant. When the blood conduit is disposed in a position requiring it to bend (such as, e.g., when the blood pump extends from the left ventricle through the aortic valve into the ascending aorta), the inside surface of the blood conduit may move closer to one side of the distal portion of the impeller, as shown in FIG. 8B. The collective features described herein and above allow for the blood pump of the present disclosure to maintain tip gap even when loads are applied to the pump, causing bending or deformation in various sections of the pump. As described above, preferential bending and flexibility in the distal tip, central portion of the scaffold, and distal shaft portion of the shaft, combined with stiffness in the inlet section and outlet section of the blood pump, allow the cantilevered impeller to maintain tip gap within the blood conduit.

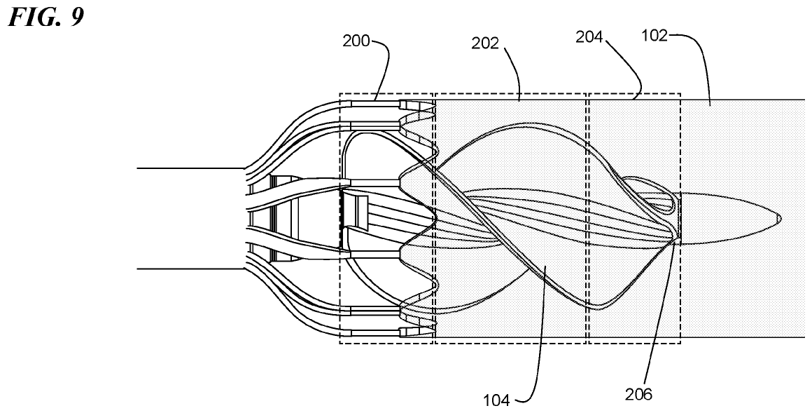

FIG. 9 shows one embodiment in which the membrane 108 and scaffold 106 are illustrated in phantom so that the impeller 104 can be seen. In this example, the impeller 104 tapers from its proximal portion 200 toward its distal portion 204, i.e., the diameter of the impeller blades decreases from the proximal portion 200 of the impeller 104 through the central portion 202 and distal portion 204 toward the distal end 206 of the impeller 104. The diameter of the blood conduit 102, on the other hand, remains substantially constant. The gap between the impeller blade tips and the blood conduit 102 therefore increases from the proximal portion 200 of the impeller toward the distal end 206 of the impeller. In one embodiment, the impeller 104 can taper or reduce in diameter only within distal portion 204 of the impeller (i.e., the impeller blade diameter is relatively constant in proximal and central portions 200 and 202. In some embodiments, the tapered sections may have different taper angles.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An expandable catheter blood pump comprising:
a blood conduit supported by a catheter, the blood conduit comprising an expandable scaffold and an impermeable membrane disposed on a portion of the expandable scaffold;
an impeller assembly rotatably supported within a proximal portion of the blood conduit;
wherein the impeller assembly is the only structure positioned within the blood conduit.

2. The blood pump of claim 1, wherein the impeller assembly includes one or more impeller blades coupled to an impeller shaft.

3. The blood pump of claim 2, wherein the impeller shaft passes through an impeller bearing assembly proximal to the one or more impeller blades.

4. The blood pump of claim 3, wherein the impeller bearing assembly is at least partially positioned within a proximal hub of the blood pump.

5. The blood pump of claim 4, further comprising a flexible drive cable coupled to a proximal end of the impeller shaft.

6. The blood pump of claim 5, wherein the impeller shaft is stiffer than the flexible drive cable.

7. The blood pump of claim 1, wherein the impeller assembly is cantilevered.

8. The blood pump of claim 1, wherein there are no structures positioned distal to the impeller assembly within the blood conduit.

9. The blood pump of claim 1, wherein the blood conduit comprises an open lumen distal to the impeller assembly.

10. The blood pump of claim 1, wherein no bearings are positioned distal of the impeller assembly.

11. The blood pump of claim 1, wherein the impeller assembly is disposed within an outlet section of the expandable scaffold.

12. The blood pump of claim 11, wherein the outlet section includes a plurality of proximal struts that form at least one opening.

13. The blood pump of claim 12, wherein the at least one opening is positioned proximal to a proximal end of the impermeable membrane.

14. The blood pump of claim 12, wherein the impeller assembly is at least partially disposed within the outlet section.

15. The blood pump of claim 11, wherein the expandable section further comprises a central portion distal to the impeller assembly.

16. The blood pump of claim 15, wherein the central portion is more flexible than the outlet section.

17. The blood pump of claim 11, wherein the catheter further comprises a distal shaft section coupled to the outlet section.

18. The blood pump of claim 17, wherein the distal shaft section is more flexible than the outlet section.

19. An expandable catheter blood pump comprising:
a blood conduit supported by a catheter, the blood conduit comprising an expandable scaffold and an impermeable membrane disposed on the expandable scaffold, wherein the expandable scaffold includes five distinct sections including:
a) a plurality of distal struts forming an inlet section,
b) a distal shroud section,
c) a central shroud section,
d) a proximal shroud section, and
e) a plurality of proximal struts forming an outlet section;
an impeller disposed in the proximal shroud section and rotatably supported by a drive cable assembly that extends through the catheter, through the outlet section, and into the proximal shroud section, wherein the drive cable assembly is more flexible proximal to the outlet section than it is through the outlet section and proximal shroud section.

20. The blood pump of claim 19, wherein the drive cable assembly does not extend distally into the central shroud section.

21. The blood pump of claim 19, wherein the impeller and the drive cable assembly are not positioned in the central shroud section, the distal shroud section, or the inlet section.

22. The blood pump of claim 19, wherein the drive cable assembly comprises a flexible drive cable coupled to a rigid impeller shaft.

23. The blood pump of claim 22, wherein the flexible drive cable is coupled to the rigid impeller shaft proximal to an impeller bearing assembly.

24. The blood pump of claim 22, wherein the flexible drive cable is more flexible than the rigid impeller shaft.

25. The blood pump of claim 19, wherein the drive cable assembly and impeller are cantilevered.

26. The blood pump of claim 19, wherein there are no structures positioned within the blood conduit except for the impeller and drive cable assembly.

27.-34. (canceled)
